(19) United States
(12) Patent Application Publication
(10) Pub. No.: US 2002/0182657 A1
(43) Pub. Date: Dec. 5, 2002

(54) Pathogen detection system for drinking water

(76) Inventor: Hubert O. Ranger, Milwaukee, WI (US)

Correspondence Address: Robert A. Van Someren, Fletcher, Yoder & Van Someren, P.O. Box 692289, Houston, TX, 77269-2289, US

(21) Appl. No.: 09/870,099

(22) Filed: May 30, 2001

(51) Int. Cl. G01N 33/53; G01N 33/554; G01N 33/569; C12Q 1/04; C12M 1/34

(52) U.S. Cl. 435/7.32; 435/34; 435/287.2

(57) ABSTRACT

A technique for detecting a pathogen in drinking water. The technique utilizes a water sampler to periodically divert samples from a drinking water supply. A detector is used to determine the presence of a pathogen in the sample and to provide a signal to an appropriate warning device.

### FIELD OF THE INVENTION

The present invention relates generally to the detection of certain biological agents, and particularly to a system of detecting pathogens in drinking water.

### BACKGROUND OF THE INVENTION

Water is an essential resource upon which our survival depends. As a result, maintaining readily available sources of drinking water is a necessity. Additionally, great emphasis has been placed on monitoring and maintaining pure drinking water to avoid the illnesses and other problems associated with the use of poor quality water as drinking water.

In recent history, substantial attention has been given to protecting drinking water supplies from trace elements and chemicals. For example, laws, regulations and public attention have been directed to reducing or eliminating lead in various water supplies by removing lead sources, such as lead water pipes that permit lead to leach into the drinking water. Multiple other elements have been regulated or otherwise controlled to reduce or eliminate their presence in sources of drinking water.

However, less attention has been given to the presence of pathogens in drinking water. This lack of attention is surprising given that pathogens can cause much more rapid effects, e.g. sickness and even death, than normally results from the slow accumulation of trace chemicals. In a variety of recorded events, illness and death has resulted from: contaminated ground water wells; the presence of cryptosporidian in city drinking water; and the presence water Esherisia Coli 0157:H7, a specific species that can cause illness when consumed with drinking water.

Various municipalities and regions combat the presence of pathogens with water treatment systems designed to remove contaminants. However, contamination of the treated water can occur downstream for a variety of reasons. For example, certain sanitation procedures, such as chlorination, may produce nutrients for the ever present spores of pathogens. Additionally, water standing in holding ponds, tanks or pipes may be become media for rapidly reproducing pathogens which eventually move downstream with the water to an unknowing end user. It would in the end user's interest to have a simple, easy-to-use system of continually checking for the presence of one or more pathogens in drinking water at the point of use.

### SUMMARY OF THE INVENTION

The present invention features a technique for monitoring the presence of pathogens at an end user location. One exemplary embodiment of this system comprises a water supply line able to supply potable water to a consumer. The system can be used in a variety of structures, such as a residential structure. The system may include a water sampler coupled in fluid communication with the water supply line to draw a plurality of sequential samples of water from the supply line. A detector is coupled to the water sampler to detect whether a pathogen exists within a given sample. Additionally, an indicator, such as visual and/or audible indicator is coupled to the detector to provide the consumer with notice of a pathogen or pathogens in the water.

### BRIEF DESCRIPTION OF THE DRAWINGS

The invention will hereafter be described with reference to the accompanying drawings, wherein like reference numerals denote like element, and:

FIG. 1 is a schematic representation of a system according to one embodiment of the present invention in which a detector is utilized within a structure;

FIG. 2 is an illustration of the system according to another embodiment of the present invention in which a consumer detection and indicator device is combined with a residential structure to provide notice of a given pathogen or pathogens;

FIG. 3 is a schematic illustration of the detector and indicator system of FIGS. 1 and 2 coupled to an exemplary water line;

FIG. 4 is a more detailed schematic illustration of the device of FIG. 3; and

FIG. 5 is a circuit diagram of one exemplary circuit than can be used to provide an indication of the presence of a pathogen.

### DETAILED DESCRIPTION OF EXEMPLARY EMBODIMENTS

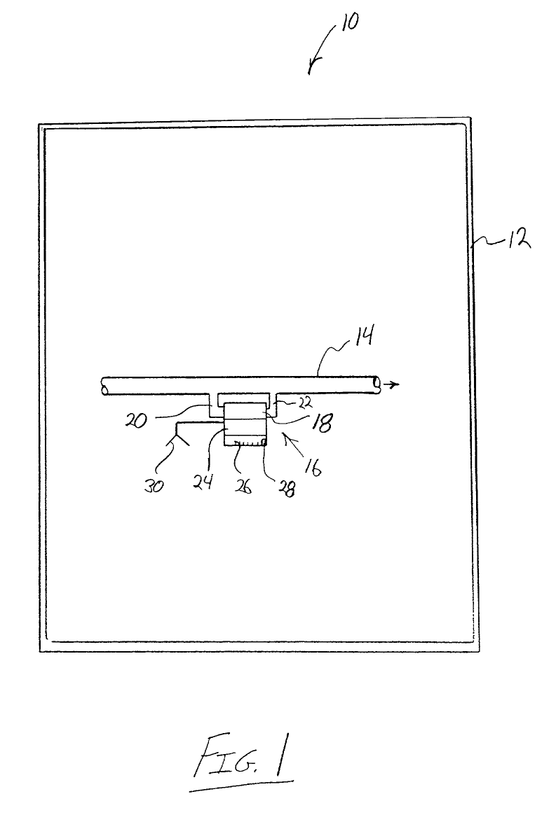

Referring generally to FIG. 1, a schematic representation of a system 10 is illustrated according to one embodiment of the present invention. In this schematic illustration, a structure 12 includes a water line 14 for delivering potable water to a desired location for drinking or other uses. Structure 12 represents a variety of potential structures having water lines to supply water for consumption. Exemplary structures, include residences, work places, public drinking fountains, RVs, and a variety of other structures used or inhabited by consumers of drinking water.

In the illustrated embodiment, a pathogen detection system 16 is shown. The exemplary pathogen detection system 16 comprises a water sampling device 18 coupled in fluid communication with water line 14 by, for example, an appropriate inlet flow path 20 and an outlet flow path 22. In this embodiment, outlet flow path 22 directs samples intaken through inlet flow path 20 back to water line 14. However, the water samples also could be directed to waste following testing.

The pathogen detection system 16 further includes a detector 24 designed to detect the presence of one or more pathogens in the sample or samples intaken by water sampler 18. Additionally, pathogen detection system 16 comprises an indicator 26 that provides, for example, visual and/or audible warnings upon the detection of a pathogen. The specific configuration and design of pathogen detection system 16 may vary substantially depending on the particular application, space constraints, structure design, etc. However, the overall structure preferably has a relatively small “footprint,” e.g. less than 10 inches in length, to permit easy use in a variety of consumer environments.

An exemplary detection system may be turned “on” or “off” by an appropriate power switch 28 readily available to the consumer. In designs that require electric power, power may be provided by a conventional power source 30, such as battery, electrical outlet, or other power source available within structure 12.

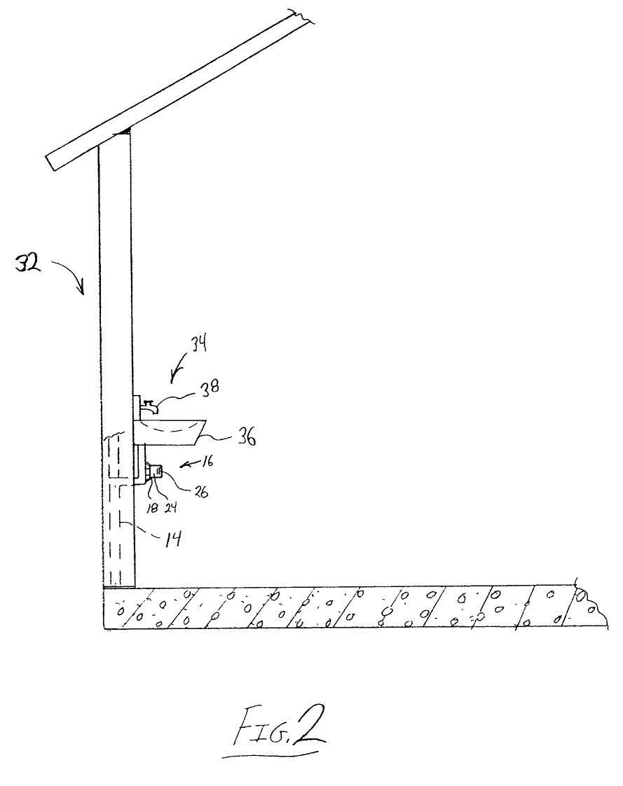

FIG. 2 illustrates an exemplary use of pathogen detection system 16 in a residential dwelling 32. Water line 14 supplies water from a well or a municipal source to various locations 34 within the house 32. Water line 14 may be directed to, for example, a sink 36 into which water is dispensed through an appropriate outlet, such as a faucet 38.

Water sampler 18 is coupled to water line 14; detector 24 is configured to detect the presence of a given level of a pathogen or pathogens; and indicator 26 is located to provide appropriate warning to an individual proximate sink 36. If the user is provided with visual indication, indicator 26 is generally placed in a readily viewable position. However, the use of an audible indicator may permit placement of indicator 26 in a hidden location.

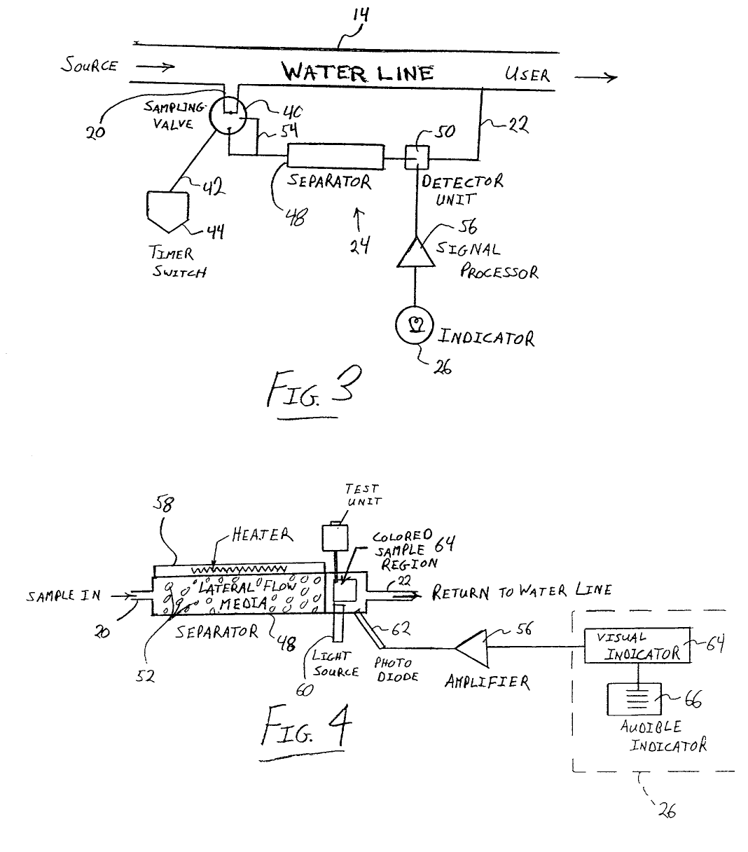

Referring generally to FIG. 3, a more detailed illustration of pathogen detection system 16 is provided. In this example, water sampler 18 comprises a sampling valve 40 that controls the flow of samples along inlet flowpath 20. Sampling valve 40 may be an automatically controlled valve. One example is an electronically controlled valve that is opened and closed on a basis established by an appropriate electronic circuit 42 having a timer switch 44. Thus, sequential samples may be taken from water line 14 on, for example, a periodic basis. Timer switch 44 may be manually adjustable to give the consumer control over the sampling, e.g. by adjusting the frequency of sampling.

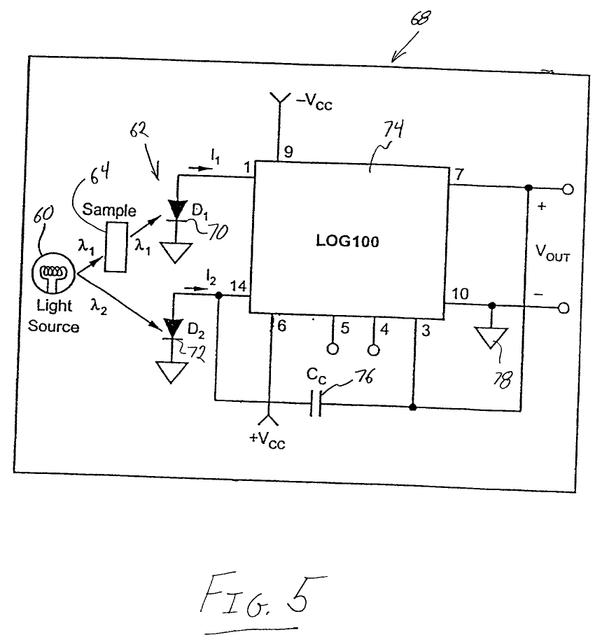

The samples are delivered to detector 24 which typically includes a separator 48 and a detector unit 50. In this embodiment, detector 24 comprises an immunochematographic device with an antigen-antibody complex. The exemplary separator 48 may be filled with an appropriate media 52 (see FIG. 4), such as a bead technology available under the tradename ATRO-Sphere™ distributed by Idexx Laboratories.

As with automated flow chemistry analyzers, the water sample is allowed to disperse through reactant media 52. Reactant media 52 is selected to react with a given pathogen or pathogens which, when present, cause the creation of an antigen. As the water sample flows to separator 48, a visible color is introduced into the sample at an upstream position via, for example, a color injection line 54 that may be controlled by an appropriate valve. In the illustrated embodiment, sampling valve 40 is a multiport valve that also controls the flow of the color sample through color injection line 54.

The color sample flows, along with the water sample, into separator 48 where it is allowed to contact the antigen which, if present, reacts to form another visible color. This latter color is detectable by detector unit 50 which outputs an appropriate signal to indicator 26 so that a visual and/or audible warning may be provided. The signal output by detector unit 50 can be modified, if necessary, by an appropriate signal processor 56. If the subject pathogen is not present in the water sample, no color change occurs and detector unit 50 provides no visual or audible alarm.

In some applications, it may be necessary or desirable to heat the water sample and color introduced into separator 48 for a period of time. An appropriate heater 58 can be combined with separator 48 to provide the necessary heat for incubation. For example, a thermostatically controlled resistance heater may be provided to maintain the sample at a desired temperature for the preset amount of time. In an exemplary application, the sample temperature is held at approximately 43° C. for about 30 minutes to permit sufficient color change in the presence of the subject pathogen.

As illustrated best in FIG. 4, detector unit 50 may utilize a light source 60 and a photoelectric cell 62 to determine whether the color change occurs. Typically, light source 60 is selected to produce light at a desired wavelength that is absorbed by the new color once the color change occurs. Photoelectric cell 62 is able to detect the light from source 60, but in the presence of the new color (color indicative of the presence of a pathogen) the light is blocked from reaching photoelectric cell 62.

For example, a yellow color sample may be introduced into separator 48 along with the water sample. In the exemplary embodiment described above, the yellow sample turns to blue when a sufficient level of the subject pathogen is present in the water sample. Light source 60 and photoelectric cell 62, e.g. a photodiode, are positioned about a colored sample region 64 of detector unit 50. When no pathogen is present, the material in colored sample region 64 does not turn blue and light is transmitted through the sample to photoelectric cell 62. If the pathogen is present, however, the sample in colored sample region 64 turns blue and absorbs the preselected wavelength light emitted from light source 60. When this light is not transmitted through colored sample region 64 to photoelectric cell 62, a signal is output to signal processor 56, e.g. an appropriate amplifier, which can amplify the signal provided to indicator 26. As described above, exemplary indicators include visual indicators 64 and/or audible indicators 66. It should also be noted that the light may be directed to photoelectric cell 62 by either direct transmission or reflectance.

Also, various circuitry may be used in detecting the presence of a pathogen, as known and used with automated flow chemistry analyzers. However, one exemplary type of circuitry 68 for the application described above is illustrated in FIG. 5. In this example, light source 60 is positioned to direct light towards colored sample region 64. On an opposite side of colored sample region 64 from light source 60 is positioned a photodiode 70 that may be or form part of photoelectric cell 62. Additionally, a second photodiode 72 is positioned directly in line with light emitted from light source 60. Thus, when the subject pathogen is present in a sample, the light from light source 60 is prevented from being transmitted through colored sample region 64 to photodiode 70.

Diodes 70 and 72 are each coupled to an appropriate logic circuit 74, such as that embodied in the LOG100 component available from Lachat Instruments. Typically, power is supplied to logic circuit 74 via appropriate terminals, e.g. −V_{cc}./+V_{cc}. Additionally, other components may be coupled to logic circuit 74 as would be apparent to one of ordinary skill in the art for a specific application, such as a capacitor 76 coupled to appropriate terminals of logic circuit 74. Logic circuit 74 also is coupled to a ground 78, and a signal is output, as represented by V_{OUT}.

The appropriate signal (V_{OUT}) is output when a predetermined condition exists at photodiodes 70 and 72. In this particular example, when a pathogen is present and colored sample region 64 has undergone the consequent color change, the transmission of light to photodiode 70 is blocked but the transmission of light to photodiode 72 is uninterrupted. Thus a signal is output to logic circuit 74 from photodiode 72 but not from photodiode 70. Under these conditions, logic circuit 74 is configured to output a signal (V_{OUT}) indicative of the presence of a pathogen. This signal is amplified via signal processor 56 and an appropriate warning is provided via indicator 26.

Contrariwise, if colored sample region 64 does not block transmission of light from light source 60, both photodiode 70 and photodiode 72 output a signal to logic circuit 74. This condition results in the output of a signal (V_{OUT}) (e.g. no voltage or low voltage) representative of a water sample that has not been contaminated with a pathogen to a reactive level. Hence, the output does not initiate an alarm.

In a modified version of the invention, system 16 may be formed as a freestanding version in which structure 12 is replaced with a portable housing. Such a unit permits the manual introduction of liquid samples to be examined qualitatively for pathogens. Such a system can be utilized at a variety of locations for testing liquid samples, such as leachate from uncooked meat, seafood or from fresh or processed fruit components. Such a portable system also could be used to test water sources found in nature or from runoff. For example, the system could be utilized in testing water runoff from cattle holding areas or from other areas where concerns arise that the runoff could lead to pollution of potable water sources. Such a device also could be used as a laboratory instrument for processing samples from external sources or for use in research to evaluate water treatment methods, such as chemical and physical treatment methods.

It will be understood that the foregoing description is of exemplary embodiments of this invention, and that the invention is not limited to the specific forms shown. For example, the pathogen detection system can be designed in a variety of configurations, but provides a device small in size that may be powered by, for example, household current or a battery. The device can readily be connected to a water source so as to perform tests at preselected intervals. Timing circuit 42, for example, may be designed to permit selection of testing frequency by a user. Additionally, a variety of components can be added or substituted into the embodiment described above to detect various desired pathogens. For example, various separators, detector units, indicators and circuitry can be utilized according to a given application. Furthermore, the scope of the present invention anticipates variations and improvements in detection methods, sensitivities and time requirements as new or improved methodology becomes available. Such improvements may comprise chemical improvements that broaden the range of pathogen types that may be detected. Such improvements also may be physical, such as improvements to the circuit or overall configuration that further reduces the size and/or cost of the invention or that improves the sensitivity of invention. These and other modifications may be made in the design and arrangement of the elements without departing from the scope of the invention as expressed in the appended claims.

What is claimed is:

1. A system for monitoring the presence of pathogens, comprising:
a water supply line able to supply potable water to a consumer;
a water sampler coupled in fluid communication with the water supply line to automatically draw a plurality of sequential samples of water from the water supply line;
a detector coupled to the water sampler, the detector being able to detect whether a pathogen exists within a sequential sample; and
an indicator coupled to the detector to provide notice of the presence of the pathogen.

2. The system as recited in claim 1, wherein the water supple line is a residential water supply line.

3. The system as recited in claim 1, wherein the water sampler comprises a valve that may be opened to permit outflow of a sample stream from the water supply line.

4. The system as recited in claim 3, wherein the valve comprises an electronically controlled valve.

5. The system as recited in claim 4, wherein the valve is opened automatically on a periodic basis.

6. The system as recited in claim 1, wherein the detector comprises an immunochematographic device with an antigen-antibody complex.

7. The system as recited in claim 6, wherein the detector comprises a photo-electric cell.

8. The system as recited in claim 1, wherein the detector is configured to detect occurrence of a color change in a sequential sample.

9. The system as recited in claim 1, wherein the indicator comprises a visual indicator.

10. The system as recited in claim 1, wherein the indicator comprises an audible indicator.

11. A method for warning a user of contamination of a drinking water supply, comprising:
connecting a sample flow path to a potable water supply line used to supply the potable water to an individual;
diverting a water sample into the sample flow path;
testing the water sample for a pathogen; and
providing an indication of whether the pathogen exists in the water sample.

12. The method as recited in claim 11, wherein connecting comprises connecting the sample flow path to a residential water supply line.

13. The method as recited in claim 11, wherein diverting comprises opening a valve.

14. The method as recited in claim 13, wherein diverting comprises diverting a plurality of samples on a periodic basis.

15. The method as recited in claim 11, wherein testing comprises directing the water sample into a sample cell.

16. The method as recited in claim 11, wherein testing comprises utilizing an immunochematographic device having an antigen-antibody complex.

17. The method as recited in claim 11, wherein testing comprises checking for a color change to the water sample.

18. The method as recited in claim 11, wherein providing comprises providing an audible indication.

19. A residential system for detecting a pathogen in a water supply, comprising:
a residential structure;
a water supply system disposed in the residential structure; and
a pathogen detection system fluidically coupled with the water supply system wherein the pathogen detection system automatically diverts and isolates samples of water from the water supply system to test for the pathogen.

20. The residential system as recited in claim 19, wherein the pathogen detection system continually diverts and isolates samples of water.

21. The residential system as recited in claim 20, wherein the pathogen detection system comprises a valve to selectively divert the water samples.

22. The residential system as recited in claim 21, wherein the pathogen detection system comprises an immunochematographic device with an antigen-antibody complex.

23. The residential system as recited in claim 22, wherein the pathogen detection system comprises a photo-electric cell.

24. The residential system as recited in claim 19, wherein the pathogen detection system is configured to detect occurrence of a color change in a sequential sample.

25. The residential system as recited in claim 22, wherein the pathogen detection system further comprises an audible indicator to indicate the presence of the pathogen.

26. The residential system as recited in claim 22, wherein the pathogen detection system further comprises an visible indicator to indicate the presence of the pathogen.
